(19) United States
(12) Patent Application Publication
(10) Pub. No.: US 2004/0058989 A1
(43) Pub. Date: Mar. 25, 2004

(54) Pharmaceutical composition containing citalopram

(75) Inventors: Ken Liljegren, Vaerlose (DK); Per Holm, Vanlose (DK)

Correspondence Address: DARBY & DARBY P.C., P. O. BOX 5257, NEW YORK, NY, 10150-5257, US

(73) Assignee: H. Lundbeck A/S, Copenhagen-Valby (DK)

(21) Appl. No.: 10/619,743

(22) Filed: Jul. 1, 2003

Related U.S. Application Data
(63) Continuation of application No. PCT/DK02/00003, filed on Jan. 3, 2002.

(30) Foreign Application Priority Data
Jan. 5, 2001 (DK) PA 2001 00016

(51) Int. Cl. A61K 31/343

(52) U.S. Cl. 514/469

(57) ABSTRACT

A solid unit dosage form comprising citalopram which is prepared by a process comprising a step wherein citalopram base or a pharmaceutically acceptable salt thereof and optionally pharmaceutically acceptable excipients is roller compacted.

This application is a continuation of International application no. PCT/DKO 2/00003, filed Jan. 3, 2002. The prior application is hereby incorporated by reference, in its entirety.

The present invention relates to a novel pharmaceutical composition containing citalopram, 1-[3-dimethylamino)propyl]-1-(4-fluorophenyl)-1,3-dlhydro-5-isobenzo-furancarbonitrile.

### BACKGROUND OF THE INVENTION

Citalopram is a well-known antidepressant drug that has the following structure:

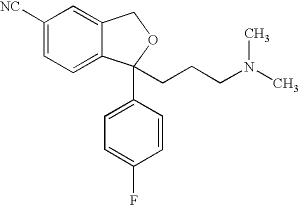

It is a selective, centrally active serotonin (5-hydroxytryptamine; 5-HT) reuptake inhibitor, accordingly having antidepressant activities.

Citalopram was first disclosed in DE 2,657,013, corresponding to U.S. Pat. No. 4,136,193. This patent publication describes the preparation of citalopram by one method and outlines a further method which may be used for preparing citalopram. The citalopram prepared was isolated in crystalline. form as the oxalate, the hydrobromide and the hydrochloride salt, respectively. Furthermore, the citalopram base was obtained as an oil (B.P. 175° C./0.03 mmHg). The publication also outlines the manufacture of tablets containing salts of citalopram. Citalopram is marketed as the hydrobromide and the hydrochloride, respectively.

Manufacture of crystalline citalopram base is disclosed in co-pending DK 2000 00402. This patent publication describes the preparation of crystalline citalopram base and the use of crystalline citalopram base as an intermediate in the purification of crude citalopram hydrobromide into pure citalopram hydrobromide. The publication also outlines the manufacture of tablets containing citalopram base.

Citalopram is marketed in a number of countries as a tablet prepared by compression of wet-granulated citalopram hydrobromide, lactose and other excipients.

It is well-recognised that preparation of tablets with a reproducible composition requires that all the dry ingredients have good flow properties. In cases, where the active ingredient has good flow properties, tablets can be prepared by direct compression of the ingredients. However, in many cases, where the particle ;size of the active substance is small, the active substance is cohesive or has poor flow properties.

Further, active substances with a small particle size mixed with excipients having a larger particle size will typically segregate or de-mix during the tabletting process.

The problems of small particle size, poor flowability and segregation are conventionally solved by enlarging the particle size of the active substance, usually by granulation of the active ingredient either alone or in combination with a filler and/or other conventional tablet ingredients.

One such granulation method is the “wet” granulation process. Using this method, the dry solids (active ingredients, filler, binder etc.) are blended and moistened with water or another wetting agent (e.g. an alcohol) and agglomerates or granules are built up of the moistened solids. Wet massing is continued until a desired homogenous particle size has been achieved whereupon the granulated product is dried.

An alternative to the “wet” granulation method is the “melt” granulation, which is also known as the “thermal plastic” granulation process, where a low melting solid is used as the granulation agent. Initially, the dry solids are blended and heated until the binder melts. As the binder is liquefied and spreads over the surface of the particles, the particles will adhere to each other and form granules. The binder solidifies upon cooling forming a dry granular product.

Wet granulation as well as melt granulation are energy intensive unit operations requiring complicated and expensive equipment as well as technical skill.

The process used for the preparation of citalopram hydrobromide results in a product with a very small particle size around 2-20 μm that, as many other particulate products with a small particle size, has very poor flow properties. Thus, in order to achieve appropriate dosing of the citalopram during tabletting, it is considered necessary to make a granulate of citalopram with larger particle size and improved flow properties.

The citalopram tablet that is marketed is a tablet made from fluid-bed dried, wet-granulated citalopram hydrobromide with various excipients.

A third size enlargement method is roller compaction where the size enlargement is done by mechanical means. Using this method, the dry solids are compressed between two rollers resulting in a sheet which subsequently is broken down into a granulate by mechanical means such as a rotating mill and oscillating screens.

The integration of the granulation into one apparatus in roller compaction is difficult to control and tends to cause very broad or even bimodal particle size distributions. Broad or bimodal particle size distributions often have adverse effects, such as poor flow characteristics, segregation, de-mixing and the like, hampering the later stages of the formulation of a pharmaceutical acceptable solid unit dosage form with constant composition.

In view of the fact that roller compaction requires fewer process steps, is much less time consuming and cheaper than the processes involving wet or melt granulation, there is a desire for a process for roller compaction of citalopram hydrobromide.

The obstacles that hitherto have hindered roller compaction of citalopram tablets have now been circumvented.

It has, surprisingly, been found that a granulate prepared by roller compaction of essentially undiluted citalopram and having a median particle size comparable to the median particle size of the filler is useful for the manufacture of compressed tablets despite the broad or bimodal particle size distribution of the granulate.

Likewise surprising, it has been found that a granulate prepared by roller compaction of citalopram mixed with all excipients for the finished formulation except for a small amount of glidant is useful for the manufacture of compressed tablets despite the broad or bimodal particle size distribution of the granulate.

Accurate dosing in capsules may also be achieved with such roller compacted granulates.

### OBJECTS OF THE INVENTION

It is the object of the present invention to provide a novel pharmaceutical unit dosage form containing roller compacted citalopram.

A second object of the invention is to provide a capsule containing citalopram.

A third object of the invention is to provide a roller compacted granulate comprising citalopram.

A fourth object of the invention is to provide a process for roller compaction of citalopram.

### SUMMARY OF THE INVENTION

The invention then, inter alia, comprises the following alone or in combination:

A solid unit dosage form comprising citalopram prepared by roller compaction of citalopram base or a pharmaceutically acceptable salt thereof, where pharmaceutically acceptable excipients optionally may be mixed with the active ingredient before granulation, and optionally the roller compacted granulate may be mixed with extragranular pharmaceutically acceptable excipients, whereupon said granulate or mixture with extragranular excipients is compressed into a tablet or filled in a hard gelatine capsule.

A granulate comprising citalopram base or a pharmaceutically acceptable salt thereof, where said granulate is formed by roller compaction of a powder comprising citalopram base or a pharmaceutically acceptable salt thereof and optionally pharmaceutically acceptable excipients.

A method for manufacture of a granulate comprising citalopram base or a pharmaceutically acceptable salt thereof, where said method comprises roller compaction of a powder comprising citalopram base or a pharmaceutically acceptable salt thereof and optionally pharmaceutically acceptable excipients.

Citalopram can be compacted alone or optionally mixed with a small amount of glidant, such as magnesium stearate, to minimize adhesion to surfaces in the compaction equipment. Afterwards, the granulate is mixed with extragranular excipients in order to form a mixture, which can be compressed into a tablet or filled in a hard gelatine capsule.

At the other end of the scale, citalopram may be mixed with all excipients prior to compaction, or, optionally, all ingredients but a small amount of glidant, which is added after compaction. Thus, the granulate, optionally admixed with glidant, is ready for tabletting or filling in a hard gelatine capsule. All ingredients are “locked” in the granule and cannot demix.

The roller compaction of citalopram and optional pharmaceutically acceptable excipients into a granulate, which can be used in formulation of pharmaceutical acceptable solid unit dosage forms has the great advantage, that wet or melt granulation, which requires a time-consuming heating or drying step, is avoided.

As used herein, “particle size distribution” means the distribution of equivalent spherical diameters as determined by laser diffraction in a Sympatec Helos equipment. The particle size distributions for fillers and uncompacted citalopram are determined at 1 bar dispersive pressure, whereas the particle size distributions for compacted granulates are determined at 0.2 bar dispersive pressure in order to avoid deaggregation of the granules leading to erroneous results. “Median particle size”, correspondingly, means the median of said particle size distribution.

Thus, in one embodiment of the invention, the present invention relates to a tablet prepared by compression of a mixture of roller compacted citalopram base or a pharmaceutically acceptable salt thereof and pharmaceutically acceptable excipients.

In another embodiment, the present invention relates to a capsule prepared by filling a mixture of roller compacted citalopram base or a pharmaceutically acceptable salt thereof and pharmaceutically acceptable excipients in a hard gelatine capsule.

Flow, segregation and demixing properties and, hence, the suitability of the granulates for compression into tablets or filling in hard gelatine capsules depend, besides the median particle size, on the particle size distribution.

Preferably, the solid unit dosage forms according to the invention do not contain a binder.

The solid unit dosage form according to the invention may contain 2-60% w/w active ingredient calculated as citalopram base, preferably 10-40% w/w active ingredient calculated as citalopram base, and more preferred 15-25% w/w active ingredient calculated as citalopram base. Suitably, the solid unit dosage form of the invention contains 20% w/w active ingredient calculated as citalopram base.

In one preferred embodiment of the invention, the present invention relates to a solid unit dosage form wherein the active ingredient is citalopram hydrobromide or citalopram hydrochloride. Preferably the active ingredient contained in the solid unit dosage form of the invention is citalopram hydrobromide.

In another preferred embodiment of the invention, the present invention relates to a solid unit dosage form wherein the active ingredient is citalopram base.

The solid unit dosage form according to the invention may contain a filler selected from lactose, or other sugars e.g. sorbitol, mannitol, dextrose and sucrose, calcium phosphates (dibasic, tribasic, hydrous and anhydrous), starch, modified starches, microcrystalline cellulose, calcium sulphate and/or calcium carbonate. In a preferred embodiment, the solid unit dosage form of the invention does not contain lactose. Suitably the filler is a microcrystalline cellulose such as ProSolv SMCC90 manufactured by Penwest Pharmaceuticals or Avicel PH 200 or Avicel PH 101 manufactured by FMC Corporation.

Besides the active ingredient and filler, the solid pharmaceutical unit dosage forms may include various other conventional excipients such as disintegrants, and optionally minor amounts of lubricants, colorants and sweeteners.

Lubricants used according to the invention may suitably be one or more of the following metallic stearates (magnesium, calcium, sodium), stearic acid, wax, hydrogenated vegetable oil, talc and colloidal silica.

Suitably the lubricant is magnesium stearate or calcium stearate.

Disintegrants include sodium starch glycolate, croscarmellose, crospovidone, low substituted hydroxypropylcellulose, modified cornstarch, pregelatizined starch and natural starch.

The granulate comprising the active ingredient after compaction has preferably a median particle size of at least 40 μm, more preferred in the range of 40- 250 μm, even more preferred in the range of 45- 200 μm and most preferred in the range of 50-180 μm.

The active ingredient is in the form of a powder prior to compaction. The powder preferably has a median particle size below 20 μm and more preferred below 15 μm.

The solid, pharmaceutical unit dosage form of the invention may be prepared by conventional methods using a tablet press with forced feed capability.

The filled, hard gelatine capsule of the invention may be prepared by conventional methods using a capsule filler suitable for powder filling. The crystals of a pharmaceutically acceptable salt of citalopram used in one embodiment of the invention may be produced according to methods described in U.S. Pat. No. 4,136,193.

The crystals of citalopram base used in one embodiment of the invention may be produced according to methods described in co-pending DK 2000 00402. In the following, the invention is illustrated by way of examples. However, the examples are merely intended to illustrate the invention and should not be construed as limiting.

### EXAMPLE 1

Compaction of citalopram hydrobromide

Citalopram hydrobromide.(8000 g) was mixed with Mg stearate (80 g) by conventional mixing. The mixture was compacted on an Alexanderwerk WP120×40 V roller compactor.

The parameters for the compaction were set as follows:

Roller speed: 8 rpm p1 Roller pressure: 6.5 kN/cm2 (70 bar)

Auger speed: 35 rpm

Product flow: 14 kg/h

Screens: 2.0 mm and 0.8 mm

Vacuum: On

The resulting granulate constitutes the intragranular phase in subsequent tabletting in Example 3. The granulate had the following properties:

| | | |
|---|---|---|
| Bulk density: | 0.40 | g/mL |
| Tapped density (1250 taps): | 0.52 | g/mL |
| Flowability through 15 mm orifice: | 5.3 | g/s |

The particle size distributions for the citalopram hydrobromide used as feed as well as the resulting granulate are listed in Table 1.

### EXAMPLE 2

Compaction of all ingredients, except magnesium stearate

Citalopram hydrobromide (3740 g), Kollidon VA64 (748 g) as binder and Avicel PH 101 (14209 g) as filler was mixed by conventional mixing. The mixture was compacted on an Alexanderwerk WP 200×75 V roller compactor.

The parameters for the compaction were set as follows:

Roller speed: 6 rpm

Roller pressure: 7.8 kN/cm2 (90 bar)

Auger speed: 45 rpm

Product flow: 65 kg/h

Screens: 2.0 mm and 0.8 mm (100 and 70 rpm respectively)

Vacuum: On

The resulting granulate constitutes the intragranular phase in subsequent tabletting in Example 4. The. granulate had the following properties:

| | |
|---|---|
| Bulk density: | 0.55 g/mL |
| Tapped density (1250 taps) | 0.75 g/mL |

The particle size distributions for the feed materials as well as the resulting granulate are listed in Table 1.

TABLE 1
Particle size distribution (Sympatec Helos) for citalopram hydrobromide
crystals (feed to compaction); compacted material, Examples 1 and 2; and
excipients, Kollidon VA 64, Avicel PH 101 and ProSolv SCMC90

| | Citalo- | | | | | |
|---|---|---|---|---|---|---|
| Quan- | pram | Exam- | Exam- | Kollidon | Avicel | ProSolv |
| tile | HBr | ple 1 | ple 2 | VA 64 | PH 101 | SCMC90 |
| (%) | (μm) | (μm) | (μm) | (μm) | (μm) | (μm) |
| 95 | 97.0 | 737 | 712 | — | 178 | 280 |
| 90 | 72.3 | 652 | 598 | 148 | 149 | 232 |
| 50 | 14.0 | 169 | 71.4 | 63.3 | 68.5 | 114 |
| 10 | 1.2 | 6.3 | 12.0 | 18.5 | 23.4 | 32.1 |

### EXAMPLE 3

Tabletting of compacted citalopram hydrobromide mixed with extragranular excipients.

Compacted material (5800 g) from Example 1 was mixed with silicified microcrystalline cellulose (ProSolv SMCC90) (22765 g) as filler in a Bohle PTM 200 (100 L) mixer for 3 minutes at 7 rpm. Magnesium stearate (144 g) was added as extra glidant and mixing continued for 30 seconds.

25 kg of the above mixture was tabletted on a Fette P 1200 IC tablet press at speeds of 50,000 to 125,000 tablets/hour. The granulate was fed by means of a forced feeder. Tablet core weight was 125 mg corresponding to a tablet strength of 20 mg citalopram base-equivalent.

During tabletting, samples were withdrawn at every 500 g granulate corresponding to every 4000 tablets. Tabletting ended after manufacture of 184,000 tablets.

Two tablets from each sample were assayed by a validated method using UV-absorption in an aqueous solution, thus analysing in total 92 tablets. The relative standard deviation in citalopram content was 4.4%.

### EXAMPLE 4

Tabletting of compacted mixture of citalopram hydrobromide, Kollidon VA64 and Avicel PH 101 with extragranular magnesium stearate.

Granulate from Example 2 was mixed with Mg-stearate as glidant. Mixing was performed in a Bohle PTM 200 (100 L) mixer for 30 seconds at 7 rpm.

TABLE 2
Composition of tablets

| | %-intragran. | qty (g) | % pr. tab. | mg pr. tab. |
|---|---|---|---|---|
| Intragranular phase | | | | |
| Citalopram HBr | 20.0% | 3740 | 19.9% | 25.0 |
| Kollidon VA64 | 4.0% | 748 | 4.0% | 5.0 |
| Avicel PH101 | 76.0% | 14209 | 75.6% | 95.0 |
| Extragranular phase | | | | |
| Mg-stearate | 0.5% | 90 | 0.5% | 0.6 |

18 kg of the above mixture was tabletted on a Fette P 1200 IC tablet press at speeds of 50,000 to 125,000 tablets/hour. The granulate was fed by means of a forced feeder. Tablet core weight was 125 mg corresponding to a tablet strength of 20 mg citalopram base-equivalent.

During tabletting, samples were withdrawn at every 500 g granulate corresponding to every 4000 tablets. Tabletting ended after manufacture of 124,000 tablets.

Two tablets from each sample were assayed by a validated method using UV-absorption in an aqueous solution, thus analysing in total 92 tablets. The relative standard deviation of content of citalopram base equivalent content was 1.2%.

1. A pharmaceutical product comprising a solid unit dosage form which comprises citalopram, wherein the solid unit dosage form is prepared by a process comprising a step wherein citalopram base or a pharmaceutically acceptable salt and at least one pharmaceutically acceptable excipient is roller compacted.

2. The pharmaceutical product of claim 1, wherein the citalopram base or pharmaceutically accceptable salt thereof is essentially undiluted at the roller compacting step.

3. The pharmaceutical product of claim 1, wherein the citalopram base or pharmaceutically acceptable salt thereof is mixed with essentially all the excipients at the roller compacting step.

4. The pharmaceutical product of claim 1, wherein the solid unit dosage form comprises 2-60% w/w active ingredient calculated as citalopram base.

5. The pharmaceutical product of claim 1, wherein the solid unit dosage form comprises 10-40% w/w active ingredient calculated as citalopram base.

6. The pharmaceutical product of claim 1, wherein the solid unit dosage form comprises 15-25% w/w active ingredient calculated as citalopram.

7. The pharmaceutical product of claim 1, wherein the granulate after compaction has a median particle size of at least 40 μm.

8. The pharmaceutical product. of claim 1, wherein the granulate after compaction has a median particle size of 40-250 μm.

9. The pharmaceutical product of claim 1, wherein the granulate after compaction has a median particle size of 45-200, μm.

10. The pharmaceutical product of claim 1, wherein the granulate after compaction has a median particle size of 50-180 μm.

11. The pharmaceutical product of any of claims 1-10, comprising citalopram hydrobromide or citalopram hydrochloride.
